(19) United States
(12) Patent Application Publication
(10) Pub. No.: US 2008/0076984 A1
(43) Pub. Date: Mar. 27, 2008

(54) DVT DETECTION

(76) Inventor: Nigel GOUGH, South Glamorgan (GB)

Correspondence Address: DEWITT ROSS & STEVENS, S.C.;Excelsior Financial Center, 8000 Excelsior Drive, Suite 401, Madison, WI, 53717-1914, US

(21) Appl. No.: 11/577,654

(22) PCT Filed: Oct. 19, 2005
(86) PCT No.: PCT/GB05/04022

§ 371 Date: Aug. 9, 2007

(30) Foreign Application Priority Data
Oct. 20, 2004 (GB) 0423289.8

(51) Int. Cl. A61B 5/00

(52) U.S. Cl. 600/310

(57) ABSTRACT

A device comprising a light transmission and detection system having transducers (10, 20, 7, 8), control means (5) and output means (7). The transducers are placed at various sites on the body of a patient and the light absorbed and/or reflected at these sites is measured and signals related to vasomotor activity are collected. The output can take the form of a detailed display of the vasomotor signals collected from the transducers (10, 20, 7, 8) to a simple indication of a condition present or absent. For example, the presence of a unilateral DVT can be detected by measuring the dissimilarity between two transducer signals from the soles of a patient's feet. The invention can also be used to provide an indication or not of for example, DVT and diabetic peripheral neuropathy.

The present invention relates to the detection of a range of clinical conditions including Deep Vein Thrombosis (DVT) and diabetic peripheral neuropathy, critical limb ischaemia, autonomic neural function and arterial and venous disease by the assessment of the vasomotor activity in the micro-circulation at individual sites on a body, and in particular, the detection of Deep Vein Thrombosis (DVT) and diabetic peripheral neuropathy.

Deep vein thrombosis (DVT) in the legs is a condition whereby a blood clot, develops in a vein causing partial or complete blockage of the vessel. The cause of the clot can be due to vessel damage, either from surgical procedures or trauma, or from a period of haemostasis due to prolonged periods of inactivity (e.g. long haul flight, disability) The perceivable consequences of a DVT can range from mild pain and swelling to a fatal pulmonary embolism.

Known tests used in clinical practices for the detection of DVT include imaging tests such as venography and duplex ultrasonography. Venography requires the injection of a radio opaque imaging medium and X-ray imaging requiring expert interpretation and is hazardous and uncomfortable to the patient, time consuming, expensive and not suitable for primary care or a General Practitioner (GP). Similarly, Duplex ultrasonography is a time consuming and expensive process not suitable for primary care or for GPs requiring highly skilled practitioners.

Plethysmography is a known test which is low cost, relatively quick, and is used in trained primary care or by a trained GP. However plethysmography requires the patient to exercise during the test which is not suitable for all patients and the test requires an expert operator and is not always reliable. There is also D-dimer assay test that measures the clotting agents in blood and is recommended to be used in conjunction with other tests. The plethysmography and D-dimer tests are used as a front line screening means to remove as many patients as possible without a DVT from progressing to the more onerous imaging tests of duplex ultrasonography or venography.

The invention seeks to make improvements.

Accordingly, the present invention provides a device comprising a light transmission and detection system to assess vasomotor activity in the micro-circulation at individual sites on a body for the monitoring and assessment of a range of clinical conditions including suspected DVT, diabetic peripheral neuropathy, critical limb ischaemia, autonomic neural function and arterial and venous disease.

Vasomotor activity in the micro-circulation is the continuous process of contraction and dilatation of the micro-vessels and serves several important functions including blood pressure regulation, temperature regulation, tissue oxygenation and nutrition. The control of this process is both local and systemic. Local control is activated by chemical signalling from the adjacent tissues while the systemic control originates from the autonomic sympathetic nervous system, principally for the regulation of core temperature and systemic blood pressure. The resulting local blood volume variation provides information on many of the biological processes both locally and systemically.

In a preferred embodiment, the invention comprises a light transmission and detection system including wave transducers, the wave transducers placed at one or more sites on a body, control means to measure the light absorbed and/or reflected at the or more sites and provide signals relating to the absolute value at the or more sites and/or the differential value between the sites. Preferably, the transducers are infra red wave transducers.

The present invention uses the transducers to monitor the micro-circulation blood volume variation beneath the transducer continuously. The light absorption is proportional to the volume of blood or, conversely, light reflection is inversely proportional to blood volume. For a resting patient in a stable environment, either seated or supine, the major changes of blood volume are manifestations of systemic control. Further, in the limbs, the systemic vasomotor control is symmetrical. Therefore, by placing a transducer on the sole of each foot of a healthy subject, the signal from each transducer will be similar if not identical. The presence of a unilateral DVT can be detected by measuring the dissimilarity between the two transducer signals as the distal volume of the affected leg is increased due to increased outflow resistance. This imposes altered frequency and phase characteristics in the vasomotor variation of the affected leg and therefore affects the bilateral symmetry.

In another aspect of the invention the signals received from the transducers are used in the assessment of autonomic systemic and peripheral neuropathy. Conventional systemic, autonomic function testing, analyses heart rate variability, usually derived from the ECG waveform. However, cardiac pulsation can be seen in the signal collected at most points on the skin around the body using the transducer. Therefore, heart rate variability can be derived from this signal. Analysis of the variation in the heart rate component can then be compared to the low frequency variation of the signal from the transducer, allowing a direct comparison of peripheral and systemic autonomic function. In the healthy subject both sources of variation should be similar, whereas in the patient suffering with peripheral neuropathy alone there will be a dissimilarity.

The advantages of using vasomotor activity in the feet to assess DVT, vascular disease and neurological function include the ability to use a passive test requiring no movement on the part of the patient. Preferably, the neurological function test is augmented by stress testing such as valsalva manoeuvre or mild graduation of exhalation impedance. The sites to be used on the patient's body are easily accessible, requiring low cost instruments, lower level of skill than existing tests and providing reliable results.

To date, there is little work published on the use of vasomotor activity for the assessment of clinical conditions such as those of the present invention due to the poor understanding of vasomotor activity and related biological processes. We have found that the vasomotor signal provides valuable information concerning the many biological processes occurring simultaneously within healthy and unhealthy bodies.

The invention will now be described by way of example only, with reference to the following drawings, of which:

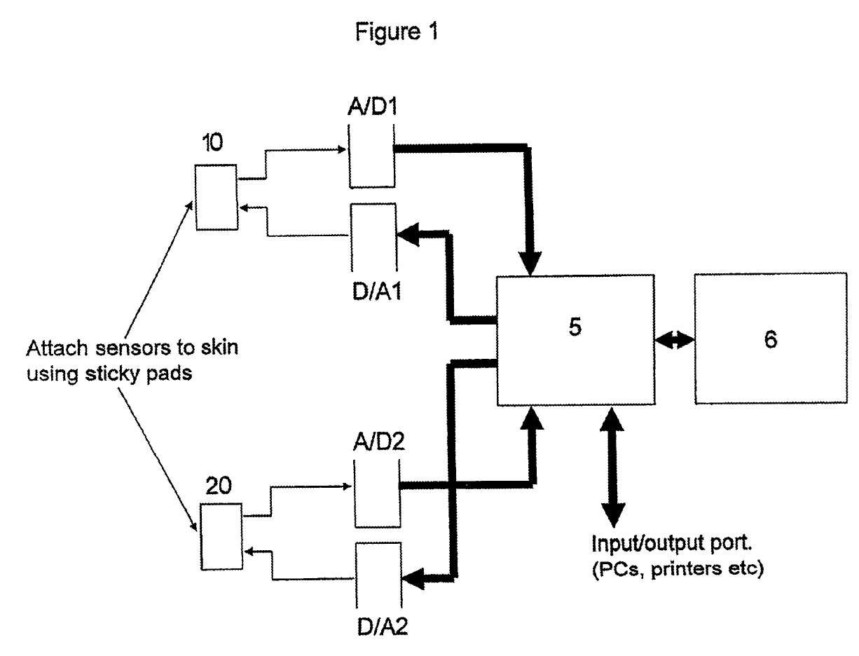

FIG. 1 shows the light transmission and detection system according to the invention;

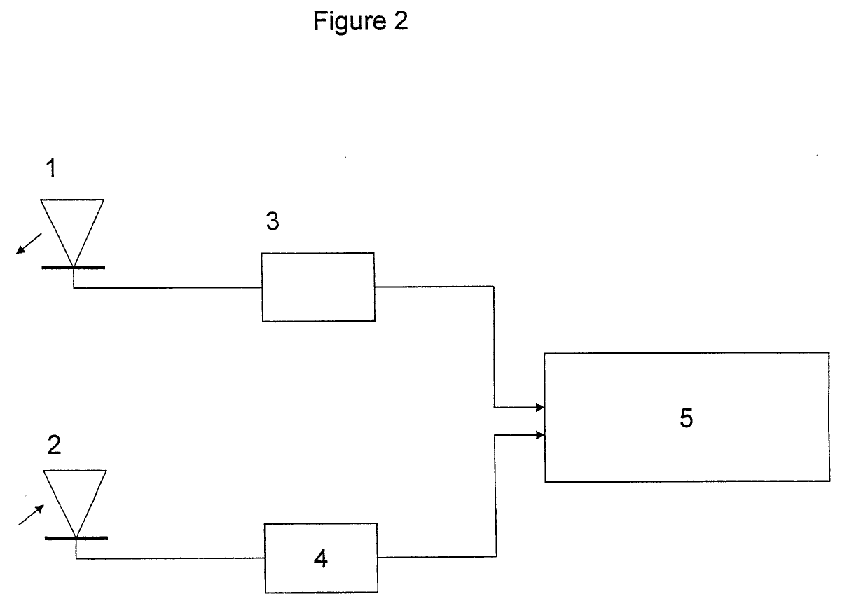

FIG. 2 shows a block diagram of the transducers in FIG. 1;

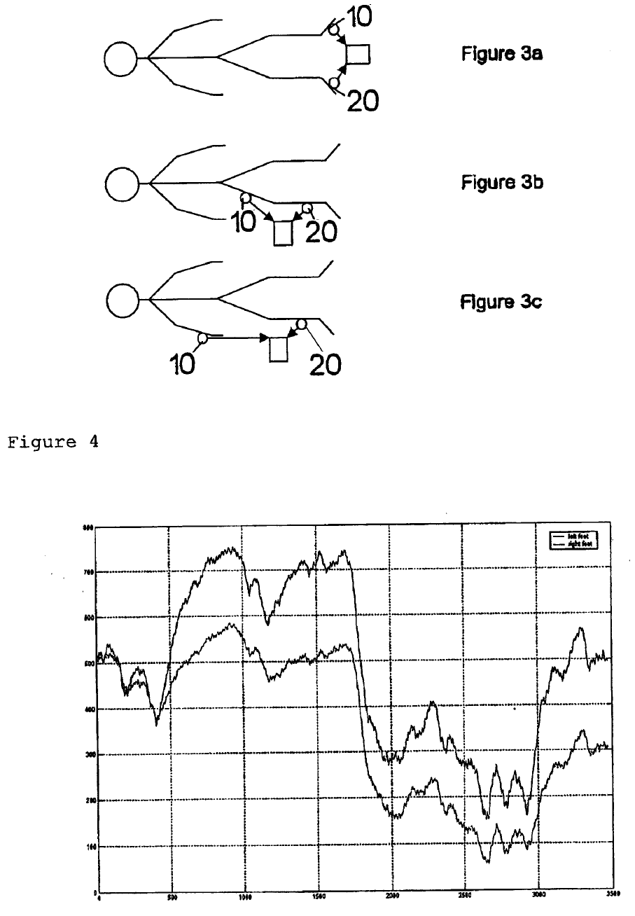

FIGS. 3a, b, c are schematic views of a preferred embodiment of the invention in FIG. 1 applied to different sites on a patient;

FIG. 4 is a signal output from the embodiment as applied in FIG. 3a;

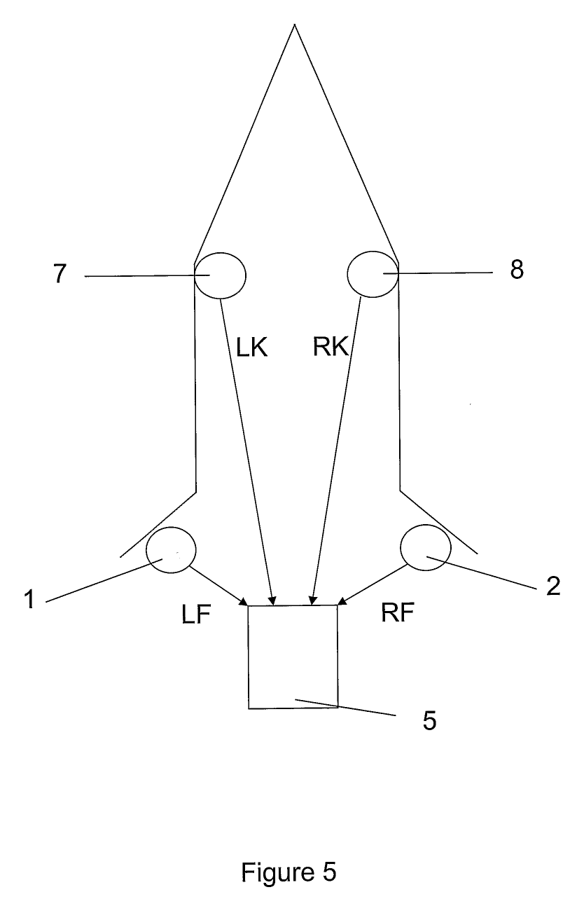

FIG. 5 shows another preferred embodiment of the invention;

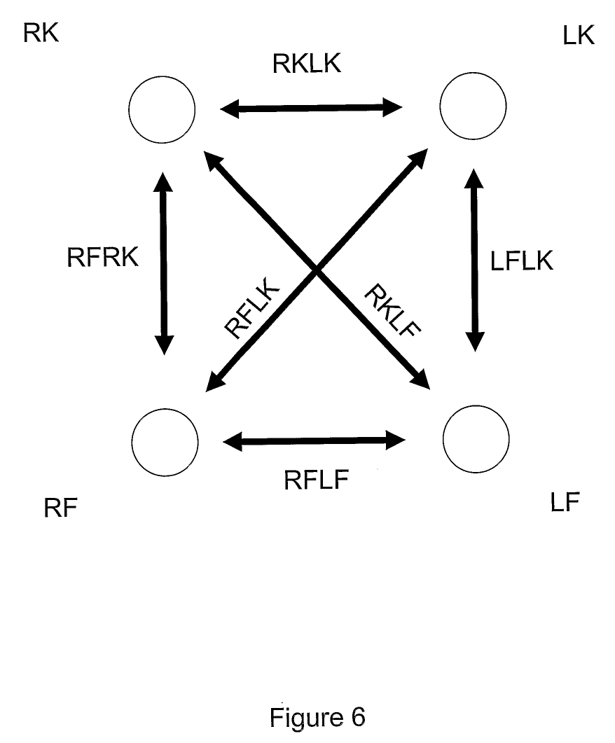

FIG. 6 shows the output from the embodiment as shown in FIG. 5 from the various sites of the legs of a patient; and

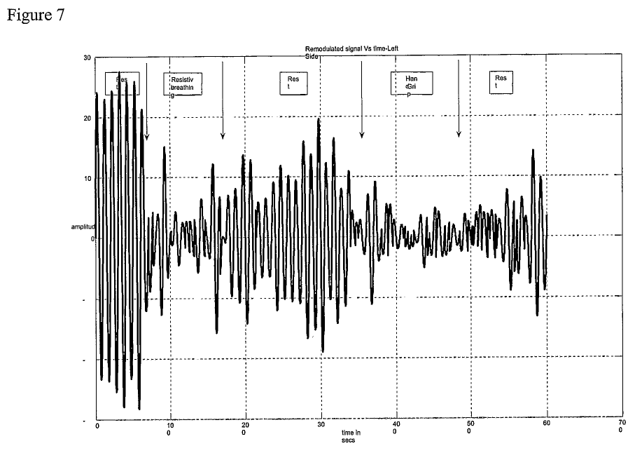

FIG. 7 shows the signal response to increased breathing impedance and hand grip.

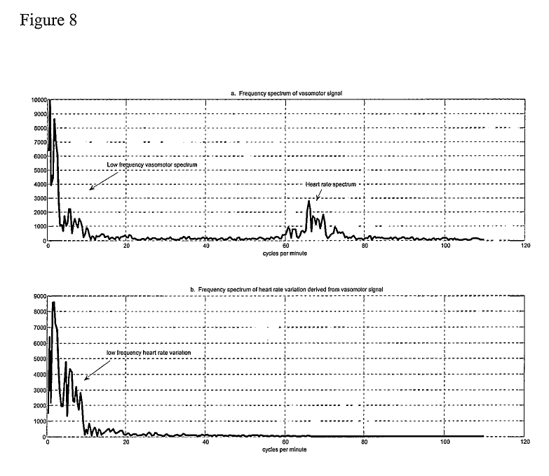

FIG. 8 shows the vasomotor signal and extraction of the heart rate variation.

Referring to FIGS. 1 and 2, the invention comprises a light transmission and detection system including transducers 1, 2 comprising an LED and photo-detector with suitable amplifiers 3, 4 as shown in FIG. 2. Once the transducers 1, 2 are attached to the skin the central control unit 5 calibrates them by driving the LED 1 with a voltage appropriate to detect a mid-scale voltage from the photo-detector 2. The photo-detector 2 signals are digitised by A/D1 and A/D2. The drive voltages for the LEDS are produced from the output of D/A1 and D/A2. Once the calibration process is complete the central control unit 5 collects data from the photo-detector 4 (FIG. 2) at a sampling rate appropriate for the application. For DVT detection a sample rate of 6 Hz is used. A user input device 6 such as a keypad and a display for output, for example an LCD screen or LED indicators or similar is used. There is also provided an input/output port for PC connection, printer or other form of data logging device.

FIGS. 3a to c show a preferred embodiment of the invention using a two channel system using two transducers 1, 2 for differential signal analysis. For the purpose of DVT detection, the transducers 1, 2 are positioned on the soles of the feet of a patient as shown in FIG. 3a. The configuration of 3b can give an indication of the approximate location of DVT. If the vasomotor signals are similar the DVT will be located in the thigh whereas if the vasomotor signals are dissimilar the DVT will be located in the calf. The arrangement in FIG. 3c indicates the pulse transit time between the upper and lower extremities and thus an indication of arterial stiffness. FIG. 4 shows the signal derived from the soles of the feet of a healthy subject using a two channel system. The signal from each transducer is similar if not identical. The presence of a unilateral DVT is detected by measuring any dissimilarity between the two signals.

The output presented to the user can take the form of a detailed display of vasomotor signals collected from the transducers 1, 2 as shown in FIG. 4 to a simple indication of a condition being present or absent. The display can be configured to the application.

The sampling rate of the transducer 1, 2 signals is such that the heart rate component can be resolved to within +/−1 ms or better if the heart rate is of interest in the assessment being performed, for example in autonomic function testing. Otherwise sampling frequencies that meet the Nyquist requirements are adequate.

The signals acquired from each transducer 1, 2 are subject to appropriate analytical algorithms. The signals are subject to amongst others complex demodulation a mathematical technique used for investigating the vasomotor activity centred at specific frequencies with a bandwidth chosen in accordance with the application, for example DVT detection. The output of the complex demodulation algorithm consists of an amplitude signal and a phase signal which when combined, produce a time varying signal modulated by both amplitude and phase with limited bandwidth, all centred on the demodulating frequency.

As well as the arrangements shown in FIGS. 3a to c, another preferred embodiment has two further transducers 7, 8 applied behind the knees for a four channel system as shown in FIG. 5. The signals are passed through the stages of signal pre-processing including filtering and DC removal followed by complex demodulation at a set of chosen frequencies, for example 8 to 30 cycles per minute. The mean absolute phase differences (MAPD) from the right foot (RF) and the left foot (LF) are calculated for each frequency to produce a spectrum RFLF(MAPD) and the RFLF(MAPD) is then used by a pattern classifier such as a pre-trained artificial neural network to provide an output on a screen that there is either “DVT PRESENT” or “DVT NOT PRESENT”.

For a four channel system as shown in FIG. 5, there will be six MAPDs as shown in FIG. 6:

Right Foot Left Foot: RFLF=mean(abs(RF(Φ)−LF(Φ))),

Right Knee Left Knee: RKLK=mean(abs(RK(Φ)−LK(Φ))),

Right Foot Right Knee: RFRK=mean(abs(RF(Φ)−RK(Φ))),

Left Foot Left Knee: LFLK=mean(abs(LF(Φ)−LK(Φ))),

Right Foot Left Knee: RFLK=mean(abs(RF(Φ)−LK(Φ))),

Right Knee Left Foot: RKLF=mean(abs(RK(Φ)−LF(Φ))),

giving six times the diagnostic information of the two channel system, described above.

In addition to detecting DVT, the present invention can monitor and assess a range of clinical conditions including diabetic peripheral neuropathy, critical limb ischaemia, autonomic neural function and arterial and venous disease.

In each of these conditions the vasomotor activity of the micro circulation possesses a unique signature which is extracted and assessed using the appropriate signal processing algorithms. These algorithms are tuned to the appropriate frequency bands determined by the clinical condition of interest. The algorithms exploit the property of vasomotor symmetry between the left and right feet and also use the similarity between the low frequency components of the vasomotor activity and the low frequency components of heart rate variation. As shown in FIG. 8, the device according to the invention, extracts from the vasomotor signal the heart rate variation and direct comparison of the simultaneous low frequency heart rate variation and the low frequency vasomotor variation provides information relating to diabetic sympathetic neuropathy, any dissimilarity between the two components indicating diabetic sympathetic neuropathy.

FIG. 7 shows the changes in vasomotor activity related to increased breathing resistance and the hand grip test of a healthy person. These tests affect systemic blood pressure and cardiac output which in turn cause neurologically mediated responses in heart rate and peripheral vasomotor activity as observed with the transducers on the soles of the feet. Any changes from the signals in FIG. 7 between the resting phase and the increased breathing resistance and the hand grip test will indicate diabetic sympathetic neuropathy since the pathology of the sympathetic nerve fibres which innovate the micro-blood vessels within the feet will cause significant change in vasomotor behaviour.

1. A device comprising a light transmission and detection system to assess vasomotor activity at individual sites on a body for the monitoring and assessment of a range of clinical conditions including suspected DVT and diabetic peripheral neuropathy.

2. A device as claimed in claim 1, wherein the device comprises a multi channel light transmission and detection system, including one or more wave transducers, the wave transducers placed at one or more sites on a body, control means to continuously measure the light absorbed at the or more sites and provide signals relating to the absolute value at the or more sites.

3. A device as claimed in claim 1, wherein the device comprises a multi channel light transmission and detection system, including one or more wave transducers, the wave transducers placed at one or more sites on a body, control means to continuously measure the light reflected at the or more sites and provide signals relating to the absolute value at the or more sites.

4. A device as claimed in claim 2, wherein the control means measure the light absorbed at the or more sites and provide signals relating to the differential value between the sites.

5. A device as claimed in claim 3, wherein the control means measure the light reflected at the or more sites and provide signals relating to the differential value between the sites.

6. A device as claimed in claim 1 wherein the signals relating to the simultaneous low frequency heart rate variation and the low frequency vasomotor variation are extracted and compared.

7. A device as claimed in claim 1 wherein the transducers are infra red wave transducers.

8. A vasomotor activity assessment device including:
a. two or more transducers, each transducer being configured for attachment to a respective portion of a body, wherein each transducer includes:
(1) alight emitter situated to emit light into the portion of the body upon which the light emitter's transducer is situated, and
(2) a light detector situated to receive light from the light emitter through the portion of the body;
b. a control unit wherein:
(1) signals from the light detectors of the transducers are received, the signals being indicative of the light received by the light detectors; and
(2) the signals from the light detectors are compared to indicate differences in blood circulation between the respective portions of the body upon which the transducers are situated.

9. The vasomotor activity assessment device of claim 8 wherein each light emitter emits light in infrared wavelengths.

10. The vasomotor activity assessment device of claim 8 further including digital to analog converters situated between the control unit and the transducers, wherein each digital to analog converter receives instruction signals from the control unit and supplies a resulting voltage to a corresponding one of the light emitters of the transducers.

11. The vasomotor activity assessment device of claim 8 further including analog to digital converters situated between the control unit and the transducers, wherein each analog to digital converter receives signals from a corresponding one of the light detectors of the transducers and supplies a resulting signal to the control unit.

12. A vasomotor activity assessment method including the steps of:
a. providing two or more transducers, each transducer being attached to a respective portion of a body, wherein each transducer includes:
(1) alight emitter situated to emit light into the portion of the body upon which the light emitter's transducer is situated, and
(2) a light detector situated to receive light from the light emitter through the portion of the body;
b. receiving signals from the light detectors of the transducers, the signals being indicative of the light received by the light detectors; and
c. comparing the signals from the light detectors to indicate differences in blood circulation between the respective portions of the body upon which the transducers are situated.

13. The vasomotor activity assessment method of claim 12 wherein the transducers are attached to the same limb in spaced relation.

14. The vasomotor activity assessment method of claim 12 wherein the transducers are attached to different limbs of the body.
